(19) United States
(12) Patent Application Publication
(10) Pub. No.: US 2024/0316065 A1
(43) Pub. Date: Sep. 26, 2024

(54) FORMULATION FOR INCREASING HAIR GROWTH AND REDUCING HAIR THINNING

(71) Applicant: Dr Ophelia Limited, London (GB)

(72) Inventor: Ophelia Veraitch, London (GB)

(73) Assignee: Dr Ophelia Limited, London (GB)

(21) Appl. No.: 18/575,619

(22) PCT Filed: Jul. 4, 2022
(86) PCT No.: PCT/GB22/51720

§ 371 Date: Dec. 29, 2023

(30) Foreign Application Priority Data
Jul. 2, 2021 (GB) 2109592.2

(51) Int. Cl. A61K 31/58; A61K 9/00; A61K 31/203; A61K 31/4045; A61K 31/506; A61K 31/5575; A61P 17/14

(52) U.S. Cl. CPC A61K 31/58; A61K 9/0014; A61K 31/203; A61K 31/4045; A61K 31/506; A61K 31/5575; A61P 17/14

(57) ABSTRACT

A formulation for use in the treatment of increasing hair thickness, increasing hair growth, reducing hair thinning and/or reducing hair loss in a subject that has received or will subsequently or concurrently receive platelet rich fibrin (PRF), said formulation comprising 0.01 wt % to 1 wt % finasteride and 0.01 wt % to 1 wt % melatonin administered to the scalp of the subject. A formulation for use in the treatment of increasing hair thickness, increasing hair growth, reducing hair thinning and/or reducing hair loss of the eyebrows and/or eyelashes of a subject, said formulation comprising 0.01 wt % to 1 wt % finasteride and 0.01 wt % to 1 wt % melatonin administered to the eyebrows and/or eyelashes of the subject.

This invention relates to a formulation comprising finasteride and melatonin, and optionally minoxidil, for increasing hair growth and reducing hair thinning in a subject, e.g. a subject that has received or will subsequently or concurrently receive platelet rich fibrin (PRF).

The present invention also relates to a formulation comprising finasteride and melatonin for increasing hair growth and reducing hair thinning in a subject that has received or will subsequently or concurrently receive platelet rich fibrin (PRF).

### BACKGROUND

The present invention may be used to treat androgenetic alopecia, telogen effluvium, alopecia areata and scarring hair loss, for example lichen planopilaris. The present invention may also be used for general cosmetic hair thickening purposes.

Androgenetic alopecia is one of the most common forms of hair thinning and loss in both men and women.

Hair thinning in androgenetic alopecia may be described as the reduction in the diameter of some or all of the hair follicles. If hair thinning is not treated, it may lead to hair loss.

Androgenetic alopecia in women, also known as female patterned hair loss, is the most common form of hair loss to affect women, with approximately 40% of women showing signs by the age of 50. Female patterned hair loss is caused by a combination of genetic and hormonal factors, and is also associated with conditions in which androgens are elevated, such as in polycystic ovarian syndrome.

Androgenetic alopecia affecting men is known as male patterned hair loss. It is the most common type of hair loss in men, affecting about 50% of men over the age of 50. Male patterned hair loss reflects a combination of inherited predisposition with increased sensitivity to the effects of dihydrotestosterone that causes scalp hair to become thinner, shorter and lighter in colour until eventually the follicles shrink and stop producing hair. Men can become aware of a receding hairline or hair thinning at any time after puberty.

Telogen effluvium relates to a marked increase in the number of hairs shed each day. This may happen due to a disturbance of the normal hair cycle, for example due to child birth, severe trauma or illness. Alopecia areata is a common cause of non-scarring hair loss. It usually causes small round patches of hair loss on the scalp, but may also affect other areas such as the beard, eyebrows, eyelashes and body hair.

Scarring hair loss, such as lichen pilanopilaris are scarring processes because the inflammation and subsequent scarring occurs where the hair follicle stem cell resides and therefore new hair follicles are usually unable to re-grow.

Topical minoxidil is one of the most commonly used treatments for androgenetic alopecia, in both men and women. Minoxidil was first introduced as a vasodilator antihypertensive. One of the side effects of minoxidil was found to be hair growth, and therefore it was developed as a topical treatment. Minoxidil is available over the counter at 2% and 5% concentrations, and 5% has been shown in the literature to be more effective than 2%. Twice daily 5% application has also been shown to be more effective than once daily 5% application. It can take 6-12 months of daily use to see an improvement in hair thinning and loss, and continued use is needed to maintain such an improvement. However, treatment with topical minoxidil does fail to achieve hair growth in some patients.

Finasteride is a selective inhibitor of the type II 5 alpha-reductase, and inhibits conversion of testosterone to the more active form dihydrotestosterone. Plasma and scalp levels of dihydrotestosterone have been found to be reduced on treatment with oral finasteride. Oral finasteride is more commonly given to men with androgenetic alopecia. However, prescribing of oral finasteride has been limited by potential side effects such as teratogenicity, effect on sexual function and mood. More recently, studies have found that topical finasteride appears to be safer and also effective.

Currently, only topical minoxidil and oral finasteride are approved by the Food and Drug Administration (FDA) and the European Medicines Agency (EMA) for the treatment of androgenetic alopecia.

Melatonin is an endogenous hormone most commonly associated with regulation of sleep. Melatonin has however long been given to Cashmere goats to promote the yield of wool, and more recently has been reported to be effective if used topically for the treatment of androgenetic alopecia.

Latanoprost is a prostaglandin analogue and is commonly used to treat increased pressure in the eye, for example ocular hypertension and open angle glaucoma.

Tretinoin is used topically for treating acne and its anti-ageing properties. It is also used to increase the penetration and therefore effectiveness of other topical treatments for the skin and hair.

This present invention seeks to provide a formulation for increasing hair growth, reducing hair thinning and promoting hair thickening by increasing the diameter of some or all of the hair follicles comprising administration to a scalp of a subject prior to, subsequently or concurrently the subject receiving platelet rich fibrin.

### BRIEF SUMMARY OF THE DISCLOSURE

In accordance with an aspect of the present invention, there is provided a cosmetic method of increasing hair thickness, increasing hair growth, reducing hair thinning and/or reducing hair loss in a subject comprising administering a formulation comprising 0.01 wt % to 1 wt % finasteride and 0.1 wt % to 1 wt % melatonin to the subject. Typically, the amount administered will be an effective amount. The formulation may further comprise 1 wt % to 12.5 wt % minoxidil.

In accordance with an aspect of the present invention, there is provided a cosmetic method of increasing hair thickness, increasing hair growth, reducing hair thinning and/or reducing hair loss in a subject that has received or will subsequently or concurrently receive platelet rich fibrin (PRF) comprising administering a formulation comprising 0.01 wt % to 1 wt % finasteride and 0.1 wt % to 1 wt % melatonin to the subject. Typically, the amount administered will be an effective amount. The formulation may further comprise 1 wt % to 12.5 wt % minoxidil.

In accordance with an aspect of the present invention, there is provided a cosmetic method of increasing hair thickness, increasing hair growth, reducing hair thinning and/or reducing hair loss in a subject that has received or will subsequently or concurrently receive platelet rich fibrin (PRF) comprising administering a formulation comprising 0.01 wt % to 1 wt % finasteride and 0.1 wt % to 1 wt % melatonin to the scalp of the subject. Typically, the amount administered will be an effective amount. The formulation may further comprise 1 wt % to 12.5 wt % minoxidil.

In accordance with another aspect of the present invention, there is provided a cosmetic method of increasing hair thickness, increasing hair growth, reducing hair thinning and/or reducing hair loss comprising administering a formulation comprising 0.01 wt % to 1 wt % finasteride and 0.01 wt % to 1 wt % melatonin to the scalp of a subject prior to, subsequently or concurrently to administering platelet rich fibrin (PRF) to the scalp of a subject. Typically, the amount administered will be an effective amount. The formulation may further comprise 1 wt % to 12.5 wt % minoxidil.

In accordance with another aspect of the present invention, there is provided a method of treatment of hair loss in a subject comprising administering a formulation comprising 0.01 wt % to 1 wt % finasteride and 0.01 wt % to 1 wt % melatonin to the subject. Typically, the amount administered will be an effective amount. The formulation may further comprise 1 wt % to 12.5 wt % minoxidil.

In accordance with another aspect of the present invention, there is provided a method of treatment of hair loss in a subject comprising administering a formulation comprising 0.01 wt % to 1 wt % finasteride and 0.01 wt % to 1 wt % melatonin to the subject prior to, subsequently or concurrently to administering platelet rich fibrin (PRF) to the subject. Typically, the amount administered will be an effective amount. The formulation may further comprise 1 wt % to 12.5 wt % minoxidil.

In accordance with another aspect of the present invention, there is provided a method of treatment of hair loss in a subject comprising administering a formulation comprising 0.01 wt % to 1 wt % finasteride and 0.01 wt % to 1 wt % melatonin to the scalp of a subject prior to, subsequently or concurrently to administering platelet rich fibrin (PRF) to the scalp of a subject. Typically, the amount administered will be an effective amount. The formulation may further comprise 1 wt % to 12.5 wt % minoxidil.

In accordance with another aspect of the present invention, there is provided a method of treatment of hair loss in a subject that has or will subsequently or concurrently receive platelet rich fibrin (PRF), the method comprising administering a formulation comprising 0.01 wt % to 1 wt % finasteride and 0.01 wt % to 1 wt % melatonin to the scalp of a subject prior to, subsequently or concurrently to administering platelet rich fibrin (PRF) to the scalp of a subject. Typically, the amount administered will be an effective amount. The formulation may further comprise 1 wt % to 12.5 wt % minoxidil.

In accordance with another aspect of the present invention, there is provided a use of a formulation comprising 0.01 wt % to 1 wt % finasteride and 0.01 wt % to 1 wt % melatonin to increase hair thickness, increase hair growth, reduce hair thinning and/or reduce hair loss in a subject. Typically, the amount administered will be an effective amount. The formulation may further comprise 1 wt % to 12.5 wt % minoxidil.

In accordance with another aspect of the present invention, there is provided a use of a formulation comprising 0.01 wt % to 1 wt % finasteride and 0.01 wt % to 1 wt % melatonin to increase hair thickness, increase hair growth, reduce hair thinning and/or reduce hair loss in a subject that has received or will subsequently or concurrently receive platelet rich fibrin (PRF). Typically, the amount administered will be an effective amount. The formulation may further comprise 1 wt % to 12.5 wt % minoxidil.

In accordance with another aspect of the present invention, there is provided a use of a formulation comprising 0.01 wt % to 1 wt % finasteride and 0.01 wt % to 1 wt % melatonin to increase hair thickness, increase hair growth, reduce hair thinning and/or reduce hair loss prior to, subsequently or concurrently to administration of platelet rich fibrin (PRF) to a scalp of a subject. Typically, the amount administered will be an effective amount. The formulation may further comprise 1 wt % to 12.5 wt % minoxidil.

In accordance with another aspect of the present invention, there is provided a formulation comprising 0.01 wt % to 1 wt % finasteride and 0.01 wt % to 1 wt % melatonin for the treatment of hair loss in a subject. Typically, the amount administered will be an effective amount. The formulation may further comprise 1 wt % to 12.5 wt % minoxidil.

In accordance with another aspect of the present invention, there is provided a formulation comprising 0.01 wt % to 1 wt % finasteride and 0.01 wt % to 1 wt % melatonin for the treatment of hair loss in a subject that has received or will subsequently or concurrently receive platelet rich fibrin (PRF). Typically, the amount administered will be an effective amount. The formulation may further comprise 1 wt % to 12.5 wt % minoxidil.

In accordance with an aspect of the present invention, there is provided a cosmetic method of increasing hair thickness, increasing hair growth, reducing hair thinning and/or reducing hair loss in a subject comprising administering a formulation comprising 1 wt % to 12.5 wt % minoxidil, 0.01 wt % to 1 wt % finasteride and 0.01 wt % to 1 wt % melatonin to the subject.

In accordance with an aspect of the present invention, there is provided a cosmetic method of increasing hair thickness, increasing hair growth, reducing hair thinning and/or reducing hair loss in a subject that has or will subsequently or concurrently receive platelet rich fibrin (PRF) comprising administering a formulation comprising 1 wt % to 12.5 wt % minoxidil, 0.01 wt % to 1 wt % finasteride and 0.01 wt % to 1 wt % melatonin to the subject.

In accordance with an aspect of the present invention, there is provided a cosmetic method of increasing hair thickness, increasing hair growth, reducing hair thinning and/or reducing hair loss in a subject that has or will subsequently or concurrently receive platelet rich fibrin (PRF) comprising administering a formulation comprising 1 wt % to 12.5 wt % minoxidil, 0.01 wt % to 1 wt % finasteride and 0.01 wt % to 1 wt % melatonin to the scalp of the subject.

In accordance with another aspect of the present invention, there is provided a cosmetic method of increasing hair thickness, increasing hair growth, reducing hair thinning and/or reducing hair loss comprising administering a formulation comprising 1 wt % to 12.5 wt % minoxidil, 0.01 wt % to 1 wt % finasteride and 0.01 wt % to 1 wt % melatonin to the scalp of a subject prior to, subsequently or concurrently to administering platelet rich fibrin (PRF) to the scalp of a subject. Typically, the amount administered will be an effective amount.

In accordance with another aspect of the present invention, there is provided a method of treatment of hair loss in a subject comprising administering a formulation comprising 1 wt % to 12.5 wt % minoxidil, 0.01 wt % to 1 wt % finasteride and 0.01 wt % to 1 wt % melatonin to the subject. Typically, the amount administered will be an effective amount.

In accordance with another aspect of the present invention, there is provided a method of treatment of hair loss in a subject comprising administering a formulation comprising 1 wt % to 12.5 wt % minoxidil, 0.01 wt % to 1 wt % finasteride and 0.01 wt % to 1 wt % melatonin to the subject prior to, subsequently or concurrently to administering platelet rich fibrin (PRF) to the subject. Typically, the amount administered will be an effective amount.

In accordance with another aspect of the present invention, there is provided a method of treatment of hair loss in a subject comprising administering a formulation comprising 1 wt % to 12.5 wt % minoxidil, 0.01 wt % to 1 wt % finasteride and 0.01 wt % to 1 wt % melatonin to the scalp of a subject prior to, subsequently or concurrently to administering platelet rich fibrin (PRF) to the scalp of a subject. Typically, the amount administered will be an effective amount.

In accordance with another aspect of the present invention, there is provided a method of treatment of hair loss in a subject that has or will subsequently or concurrently receive platelet rich fibrin (PRF), the method comprising administering a formulation comprising 1 wt % to 12.5 wt % minoxidil, 0.01 wt % to 1 wt % finasteride and 0.01 wt % to 1 wt % melatonin to the scalp of a subject. Typically, the amount administered will be an effective amount.

In accordance with another aspect of the present invention, there is provided a use of a formulation comprising 1 wt % to 12.5 wt % minoxidil, 0.01 wt % to 1 wt % finasteride and 0.01 wt % to 1 wt % melatonin to increase hair thickness, increase hair growth, reduce hair thinning and/or reduce hair loss in a subject. Typically, the amount administered will be an effective amount.

In accordance with another aspect of the present invention, there is provided a use of a formulation comprising 1 wt % to 12.5 wt % minoxidil, 0.01 wt % to 1 wt % finasteride and 0.01 wt % to 1 wt % melatonin to increase hair thickness, increase hair growth, reduce hair thinning and/or reduce hair loss in a subject that has received or will subsequently or concurrently receive platelet rich fibrin (PRF). Typically, the amount administered will be an effective amount.

In accordance with another aspect of the present invention, there is provided a use of a formulation comprising 1 wt % to 12.5 wt % minoxidil, 0.01 wt % to 1 wt % finasteride and 0.01 wt % to 1 wt % melatonin to increase hair thickness, increase hair growth, reduce hair thinning and/or reduce hair loss prior to, subsequently or concurrently to administration of platelet rich fibrin (PRF) to a scalp of a subject. Typically, the amount administered will be an effective amount.

In accordance with another aspect of the present invention, there is provided a formulation comprising 1 wt % to 12.5 wt % minoxidil, 0.01 wt % to 1 wt % finasteride and 0.01 wt % to 1 wt % melatonin for the treatment of hair loss in a subject. Typically, the amount administered will be an effective amount.

In accordance with another aspect of the present invention, there is provided a formulation comprising 1 wt % to 12.5 wt % minoxidil, 0.01 wt % to 1 wt % finasteride and 0.01 wt % to 1 wt % melatonin for the treatment of hair loss in a subject that has received or will subsequently or concurrently receive platelet rich fibrin (PRF). Typically, the amount administered will be an effective amount.

The cosmetic method, use and/or formulation for use according to any one of the aspects of the invention, wherein the formulation is administered to the subject to areas affected by hair loss. It may be that the area affected by hair loss is selected from the group consisting of the scalp, beard, eyebrows, eyelashes and body hair. It may be that the area affected by hair loss is the scalp. It may be that the area affected by hair loss is the eyebrows. It may be that the area affected by hair loss is the eyelashes.

The cosmetic method, use and/or formulation for use according to any one of the aspects of the invention, wherein the formulation is administered to the subject to areas of hair thinning. It may be that the areas of hair thinning are selected from the group consisting of the scalp, beard, eyebrows, eyelashes and body hair. It may be that the area of hair thinning is the scalp. It may be that the area of hair thinning is the eyebrows. It may be that the area of hair thinning is the eyelashes.

The cosmetic method, use and/or formulation for use according to any one of the aspects of the invention, wherein a formulation comprising 0.01 wt % to 1 wt % finasteride and 0.1 wt % to 1 wt % melatonin is administered to the scalp, eyebrows and/or eyelashes of a subject. It may be that the formulation further comprises latanoprost. Thus, it may be that a formulation comprising 0.01 wt % to 1 wt % finasteride, 0.1 wt % to 1 wt % melatonin, and 0.0005 wt % to 0.01 wt % latanoprost is administered to the scalp, eyebrows and/or eyelashes of a subject.

The cosmetic method, use and/or formulation for use according to any one of the aspects of the invention, wherein a formulation comprising 0.01 wt % to 1 wt % finasteride, 0.1 wt % to 1 wt % melatonin, and 1 wt % to 12.5 wt % minoxidil is administered to the scalp, eyebrows and/or eyelashes of a subject. It may be that the formulation further comprises latanoprost. Thus, it may be that a formulation comprising 0.01 wt % to 1 wt % finasteride, 0.1 wt % to 1 wt % melatonin, 1 wt % to 12.5 wt % minoxidil, and 0.0005 wt % to 0.01 wt % latanoprost is administered to the scalp, eyebrows and/or eyelashes of a subject.

In embodiments, the subject will not receive platelet rich fibrin (PRF). Therefore, it may be that the subject has not received and will not subsequently or concurrently receive platelet rich fibrin (PRF). It may be that the formulation is administered to the subject to areas affected by hair loss, wherein the subject will not receive platelet rich fibrin (PRF). It may be that the formulation is administered to the subject to areas of hair thinning, wherein the subject will not receive platelet rich fibrin (PRF). It may be that the formulation is administered to the eyebrows and/or eyelashes of a subject, wherein the subject will not receive platelet rich fibrin (PRF). Preferably, the formulation is administered to the eyebrows of a subject, wherein the subject will not receive platelet rich fibrin (PRF).

In embodiments, the subject may have or may subsequently or concurrently receive platelet rich fibrin (PRF). The method may comprise administering platelet rich fibrin (PRF) to the subject. The platelet rich fibrin (PRF) may be administered to the subject (for example, by subcutaneous administration or injection). Typically, the amount administered will be an effective amount.

The platelet rich fibrin (PRF) may be administered to the subject prior to, subsequently or concurrently to administering the formulation comprising 0.01 wt % to 1 wt % finasteride and 0.01 wt % to 1 wt % melatonin to the subject. Typically, the amount administered will be an effective amount.

It may be that PRF is not administered to the scalp of the subject.

It may be that the PRF is administered to the scalp of the subject. Therefore, the subject may have or may subsequently or concurrently receive platelet rich fibrin (PRF) administered to their scalp. The method may comprise administering platelet rich fibrin (PRF) to the scalp of a subject. The platelet rich fibrin (PRF) may be administered to the scalp of a subject (for example, by subcutaneous administration or injection into the scalp). Typically, the amount administered will be an effective amount.

The platelet rich fibrin (PRF) may be administered to the scalp of a subject prior to, subsequently or concurrently to administering the formulation comprising 0.01 wt % to 1 wt % finasteride and 0.01 wt % to 1 wt % melatonin to the scalp of a subject. Typically, the amount administered will be an effective amount.

The formulation may be administered to the subject daily, and the PRF treatment may be provided to the subject at any point before, during, or after treatment with the formulation. For example, the PRF treatment may be provided to the subject 1, 2, 3, 4, 5, 6, or 7 days before, 14 days before, 21 days before, or 28 days before the subject is treated with the formulation. The PRF treatment may be provided to the subject any day during treatment with the formulation. For example, the formulation may be administered to the subject daily, and the PRF treatment may be provided to the subject on day 1, day 2, day 3, day 4, day 5, day 6, or day 7 of treatment with the formulation, on day 14 of treatment with the formulation, on day 21 of treatment with the formulation, or on day 28 of treatment with the formulation. The PRF treatment may be provided to the subject any day after treatment with the formulation. For example, if the subject stops treatment with the formulation, the PRF treatment may be provided to the subject 1, 2, 3, 4, 5, 6, or 7 days after, 14 days after, 21 days after, or 28 days after the subject is treated with the formulation.

The formulation may be provided to a subject a month in advance of providing PRF treatment.

The PRF treatment may be provided to a subject a month after the subject is treated with the formulation.

For example, 10 mls of PRF in 0.1 ml aliquots may be injected in the areas of hair thinning.

Four sessions of PRF may be provided to the subject four weeks apart.

The platelet rich fibrin (PRF) may be administered to the subject prior to, subsequently or concurrently to administering the formulation comprising 1 wt % to 12.5 wt % minoxidil, 0.01 wt % to 1 wt % finasteride and 0.01 wt % to 1 wt % melatonin to the subject. Typically, the amount administered will be an effective amount.

The platelet rich fibrin (PRF) may be administered to the scalp of a subject prior to, subsequently or concurrently to administering the formulation comprising 1 wt % to 12.5 wt % minoxidil, 0.01 wt % to 1 wt % finasteride and 0.01 wt % to 1 wt % melatonin to the scalp of a subject. Typically, the amount administered will be an effective amount.

The formulation may be provided to a subject a month in advance of providing PRF treatment.

The PRF treatment may be provided to a subject a month after the subject is treated with the formulation.

For example, 10 mls of PRF in 0.1 ml aliquots may be injected in the areas of hair thinning.

Four sessions of PRF may be provided to the subject four weeks apart.

The cosmetic method, use and/or formulation for use according to any one of the aspects of the invention, wherein the hair loss is caused by a condition selected from: androgenetic alopecia, telogen effluvium, alopecia areata and scarring hair loss.

The cosmetic method, use and/or formulation for use according to any one of the aspects of the invention, wherein the hair loss is caused by a condition selected from: androgenetic alopecia, telogen effluvium, alopecia areata and lichen planopilaris.

The cosmetic method, use and/or formulation for use according to any one of the aspects of the invention, wherein the hair loss is caused by androgenetic alopecia.

The cosmetic method, use and/or formulation for use according to any one of the aspects of the invention, wherein the formulation comprises 2 wt % to 11 wt % minoxidil.

The cosmetic method, use and/or formulation for use according to any one of the aspects of the invention, wherein the formulation comprises 0.1 wt % to 1 wt % finasteride.

The cosmetic method, use and/or formulation for use according to any one of the aspects of the invention, wherein the formulation comprises 0.05 wt % to 0.5 wt % melatonin.

The cosmetic method, use and/or formulation for use according to any one of the aspects of the invention, wherein the formulation comprises 0.1 wt % to 0.2 wt % melatonin. It may be that the formulation comprises 0.1 wt % melatonin.

The cosmetic method, use and/or formulation for use according to any one of the aspects of the invention, wherein the formulation further comprises a prostaglandin analogue at an amount between 0.01 wt % to 5 wt %.

The cosmetic method, use and/or formulation for use according to any one of the aspects of the invention, wherein the prostaglandin analogue is selected from the group consisting of: travoprost, bimatoprost, tafluprost and unoprostone.

The cosmetic method, use and/or formulation for use according to any one of the aspects of the invention, wherein the prostaglandin analogue is latanoprost.

The cosmetic method, use and/or formulation for use according to any one of the aspects of the invention, wherein the formulation further comprises a retinoic acid at an amount between 0.01 wt % to 2 wt %.

The cosmetic method, use and/or formulation for use according to any one of the aspects of the invention, wherein the retinoic acid is tretinoin.

The cosmetic method, use and/or formulation for use according to any one of the aspects of the invention, wherein the retinoic acid is not tretinoin.

The cosmetic method, use and/or formulation for use according to any one of the aspects of the invention, wherein the formulation does not comprise retinoic acid. Thus, it may be that the formulation does not comprise tretinoin.

The cosmetic method, use and/or formulation for use according to any one of the aspects of the invention in the form of an ointment, lotion and/or spray for topical administration.

There is provided a formulation comprising 0.01 wt % to 1 wt % finasteride and 0.01 wt % to 1 wt % melatonin. The formulation may further comprise 1 wt % to 12.5 wt % minoxidil.

There is provided a formulation comprising 1 wt % to 12.5 wt % minoxidil, 0.01 wt % to 1 wt % finasteride and 0.01 wt % to 1 wt % melatonin.

There may also be provided a pharmaceutical composition comprising the formulation according to the invention and a pharmaceutically acceptable carrier.

Minoxidil, finasteride and melatonin are typically provided in the formulation in synergistically effective amounts.

The formulation may comprise at least 2 wt % minoxidil. The formulation may comprise at least 4 wt % minoxidil. The formulation may comprise at least 5 wt % minoxidil. The formulation may comprise no more than 11 wt % minoxidil. The formulation may comprise no more than 10 wt % minoxidil.

The formulation may comprise 2 to 11 wt % minoxidil. The formulation may comprise 3 to 7 wt % minoxidil. The formulation may comprise 4 to 6 wt % minoxidil. The formulation may comprise 8 to 12 wt % minoxidil. The formulation may comprise 9 to 11 wt % minoxidil.

The formulation may comprise about 2 wt % minoxidil. The formulation may comprise about 5 wt % minoxidil. The formulation may comprise about 10 wt % minoxidil.

The formulation may comprise at least 0.1 wt % finasteride. The formulation may comprise at least 0.2 wt % finasteride. The formulation may comprise no more than 0.3 wt % finasteride. The formulation may comprise no more than 0.75 wt % finasteride. The formulation may comprise no more than 1 wt % finasteride.

The formulation may comprise 0.1 wt % to 1 wt % finasteride. The formulation may comprise 0.2 wt % to 0.6 wt % finasteride. For example, the formulation may comprise 0.25 to 0.5 wt % finasteride.

The formulation may comprise about 0.25 wt % finasteride.

The formulation may comprise at least 0.05 wt % melatonin. For example, the formulation may comprise at least 0.1 wt % melatonin. The formulation may comprise no more than 1 wt % melatonin. The formulation may comprise no more than 0.5 wt % melatonin. The formulation may comprise no more than 0.25 wt % melatonin. The formulation may comprise no more than 0.15 wt % melatonin.

The formulation may comprise 0.05 wt % to 0.25 wt % melatonin. The formulation may comprise about 0.1 wt % melatonin.

The formulation may comprise 0.01 wt % to 1 wt % finasteride and 0.01 wt % to 0.5 wt % melatonin. For example, the formulation may comprise 0.1 wt % to 0.5 wt % finasteride and 0.05 wt % to 0.1 wt % melatonin.

The formulation may comprise 2 to 11 wt % minoxidil, 0.01 wt % to 1 wt % finasteride and 0.01 wt % to 0.5 wt % melatonin. For example, the formulation may comprise 5 to 10 wt % minoxidil, 0.1 wt % to 0.5 wt % finasteride and 0.05 wt % to 0.1 wt % melatonin.

In an embodiment, there is provided a formulation comprising 0.25 wt % finasteride and 0.1 wt % melatonin.

In an embodiment, there is provided a formulation comprising 10 wt % minoxidil, 0.25 wt % finasteride and 0.1 wt % melatonin.

In an embodiment, there is provided a formulation comprising 5 wt % minoxidil, 0.25 wt % finasteride and 0.1 wt % melatonin.

In an embodiment, there is provided a formulation comprising 2 wt % minoxidil, 0.25 wt % finasteride and 0.1 wt % melatonin.

The formulation may further comprise a prostaglandin analogue. For example, the formulation may further comprise a prostaglandin analogue selected from the group consisting of: latanoprost, travoprost, bimatoprost, tafluprost and unoprostone.

In an embodiment, the formulation further comprises latanoprost.

The formulation may further comprise a prostaglandin analogue (e.g. latanoprost) at an amount between 0.01 wt % to 5 wt %. For example, the prostaglandin analogue may be present at an amount of 0.1 wt %.

In an embodiment, the formulation further comprises 0.1 wt % latanoprost.

The formulation may further comprise a prostaglandin analogue (e.g. latanoprost) at an amount between 0.0001 wt % to 5 wt %. For example, the formulation may further comprise latanoprost at an amount between 0.0001 wt % to 5 wt %. It may be that the latanoprost is present at an amount of 0.0005 wt % to 0.01 wt %. It may be that the latanoprost is present at an amount of 0.001 wt %. It may be that the latanoprost is present at an amount of 0.005 wt %.

In an embodiment, the formulation further comprises 0.001 wt % latanoprost. Thus, it may be that the formulation comprises 0.01 wt % to 1 wt % finasteride, 0.1 wt % to 1 wt % melatonin, and 0.001 wt % latanoprost. It may be that the formulation comprises 0.01 wt % to 1 wt % finasteride, 0.1 wt % to 1 wt % melatonin, 1 wt % to 12.5 wt % minoxidil, and 0.001 wt % latanoprost.

In an embodiment, the formulation further comprises 0.005 wt % latanoprost. Thus, it may be that the formulation comprises 0.01 wt % to 1 wt % finasteride, 0.1 wt % to 1 wt % melatonin, and 0.005 wt % latanoprost. It may be that the formulation comprises 0.01 wt % to 1 wt % finasteride, 0.1 wt % to 1 wt % melatonin, 1 wt % to 12.5 wt % minoxidil, and 0.005 wt % latanoprost.

The formulation may also further comprise a retinoic acid. For example, the formulation may further comprise tretinoin.

The formulation may further comprise a retinoic acid (e.g. tretinoin) at an amount between 0.01 wt % to 2 wt %. For example, the retinoic acid may be present at an amount of 0.01 wt %.

In an embodiment, the formulation further comprises 0.01% tretinoin.

In an embodiment, there is provided a formulation further comprising latanoprost and tretinoin.

In an embodiment, there is provided a formulation further comprising latanoprost at an amount between 0.01 wt % to 5 wt % and tretinoin at an amount between 0.01 wt % to 2 wt %. In an embodiment, there is provided a formulation comprising 5 wt % minoxidil, 0.25 wt % finasteride, 0.1 wt % melatonin, 0.1 wt % latanoprost and 0.01 wt % tretinoin.

In an embodiment, there is provided a formulation further comprising latanoprost at an amount between 0.0001 wt % to 5 wt % and tretinoin at an amount between 0.01 wt % to 2 wt %. It may be that the formulation further comprises latanoprost at an amount between 0.0005 wt % to 0.01 wt % and tretinoin at an amount between 0.01 wt % to 2 wt %.

In an embodiment, the formulation further comprises 0.001 wt % latanoprost and tretinoin at an amount between 0.01 wt % to 2 wt %. Thus, it may be that the formulation comprises 0.01 wt % to 1 wt % finasteride, 0.1 wt % to 1 wt % melatonin, 0.01 wt % to 2 wt % tretinoin, and 0.001 wt % latanoprost. It may be that the formulation comprises 0.01 wt % to 1 wt % finasteride, 0.1 wt % to 1 wt % melatonin, 1 wt % to 12.5 wt % minoxidil, 0.01 wt % to 2 wt % tretinoin, and 0.001 wt % latanoprost.

In an embodiment, the formulation further comprises 0.005 wt % latanoprost and tretinoin at an amount between 0.01 wt % to 2 wt %. Thus, it may be that the formulation comprises 0.01 wt % to 1 wt % finasteride, 0.1 wt % to 1 wt % melatonin, 0.01 wt % to 2 wt % tretinoin, and 0.005 wt % latanoprost. It may be that the formulation comprises 0.01 wt % to 1 wt % finasteride, 0.1 wt % to 1 wt % melatonin, 1 wt % to 12.5 wt % minoxidil, 0.01 wt % to 2 wt % tretinoin, and 0.005 wt % latanoprost.

The formulation may further comprise ketoconazole and/or spironolactone.

The formulation may be a pharmaceutical composition.

Typically, the subject is a human subject.

The human subject may be male.

The human subject may be female. However, the formulation is not suitable for pregnant females.

The formulation may be suitable for all ages of human subject. However, the human subject may be over 18 years old.

The formulation will typically be administered topically. The formulation will typically be for topical administration.

The formulation may be administered topically to the subject to areas affected by hair loss. The formulation may be administered topically to the subject in areas of hair thinning. The formulation may be administered topically to the scalp of a human subject.

The formulation may be administered topically to the eyebrows of a human subject. The formulation may be administered topically to the eyelashes of a human subject.

The formulation may be administered between one and two times a day. For example, the formulation may be administered once a day. The formulation may be administered twice a day.

In an embodiment, the formulation may be administered topically to the scalp of a human subject once a day. Preferably, the formulation may be administered topically to the scalp of a human subject at night.

An amount between 0.5 ml and 3 ml of the formulation may be administered topically to the scalp of the human subject. An amount of about 1 ml of the formulation may be administered topically to the scalp of the human subject.

For example, an amount of 1 ml of the formulation may be administered topically to the scalp of the human subject once a day at night.

In an embodiment, the formulation may be administered topically to the eyebrows and/or eyelashes of a human subject once a day. Preferably, the formulation may be administered topically to the eyebrows and/or eyelashes of a human subject at night.

An amount between 0.5 ml and 3 ml of the formulation may be administered topically to the eyebrows and/or eyelashes of the human subject.

For example, an amount of 1 ml of the formulation may be administered topically to the eyebrows and/or eyelashes of the human subject once a day at night.

The hair loss may be caused by a disorder selected from androgenetic alopecia, alopecia areata, telogen effluvium, scarring hair loss (such as lichen planopilaris) and any other medical cause of hair loss. The hair loss may be caused by androgenetic alopecia.

The formulation may be used for hair thickening (i.e. increasing the diameter of the hair follicle) and increasing hair growth.

The formulation may be used for hair thickening (i.e. increasing the diameter of the hair follicle) and increasing hair growth in the absence of any hair or scalp disorder, such as androgenetic alopecia.

### BRIEF DESCRIPTION OF THE DRAWINGS

Embodiments of the invention are further described hereinafter with reference to the accompanying drawings, in which:

FIG. 1 shows a photograph (A) of patient 1 from Table 1 with androgenetic alopecia before treatment and photograph (B) 3 months after the first PRF treatment.

FIG. 2 shows a photograph (A) of patient 2 from Table 1 with androgenetic alopecia before treatment and photograph (B) 6 weeks after the first PRF treatment.

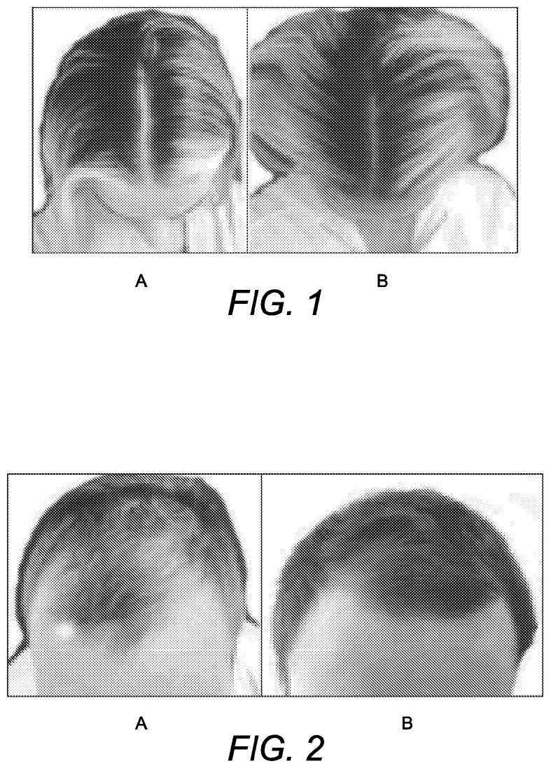

### DETAILED DESCRIPTION

Platelet rich fibrin (PRF) is a second-generation platelet-rich plasma where autologous platelets are present in a complex fibrin matrix to accelerate healing of tissue.

The iPRF Choukroun method may be used.

A sample of blood is taken from the subject and centrifuged (for example using a centrifuge for from 3 to 8 minutes at 1100-1400 revolutions per minute (e.g. 5 minutes at 1200 revolutions per minute to provide three layers consisting of a top layer of platelet poor plasma, middle layer of platelet rich fibrin clot and a bottom layer of red blood cells. The platelet rich fibrin layer may then be removed and the layer of red blood cells detached.

The platelet rich fibrin may be administered to the scalp of a subject through subcutaneous administration or injection.

For example, four vials of blood may be taken from a subject and using the iPRF Choukroun method, 10 mls of PRF in 0.1 ml aliquots may be injected into the areas of hair thinning in the scalp.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### Formulations

The formulations of the invention comprise minoxidil, finasteride, and melatonin. They will typically comprise other non-active ingredients. Formulation approaches that are useful for topical application of active compounds to the human scalp are well known to the person skilled in the art.

The formulation may further comprise an organic solvent, e.g. an alcohol. The formulation may comprise ethanol. Additionally or alternatively, the formulation may comprise water.

The formulation may further comprise an oil. For example, the formulation may further comprise coconut oil.

The formulation may be in the form of an ointment, lotion, tonic, foam and/or spray.

In an embodiment, the formulation may be in the form of a lotion. The formulation may be in the form of a foam. The formulation may be in the form of a tonic.

### EXAMPLES

### Example 1

Trials including five female and one male patients with androgenetic alopecia using a treatment protocol of (a) finasteride and melatonin or (b) minoxidil, finasteride and melatonin formulation in the form of a tonic every night in conjunction with a course of platelet rich fibrin (PRF) have been conducted.

A formulation of 0.25% finasteride and 0.1% melatonin according to the present invention is a potent topical treatment to increase hair growth and/or reduce hair thinning.

A formulation of 10% minoxidil, 0.25% finasteride and 0.1% melatonin according to the present invention is a potent topical treatment to increase hair growth and/or reduce hair thinning.

A formulation of 5% minoxidil, 0.25% finasteride and 0.1% melatonin according to the present invention is a potent topical treatment to increase hair growth and/or reduce hair thinning.

The combination of 10% minoxidil, 0.25% finasteride and 0.1% melatonin was found to be more effective in combination than when each agent was used as single agent treatment. Application of this formulation of 10% minoxidil, 0.25% finasteride and 0.1% melatonin was required only once a day. This is preferable to the usual twice daily 5% minoxidil that is usually recommended.

The formulation of 0.25% finasteride and 0.1% melatonin was provided as a topical hair tonic. Non active ingredients included ethanol, benzyl alcohol, glycerin, and purified water.

The formulation of 10% minoxidil, 0.25% finasteride and 0.1% melatonin was provided as a topical hair tonic. Non active ingredients included ethanol, benzyl alcohol, glycerin, and purified water.

The formulation of 5% minoxidil, 0.25% finasteride and 0.1% melatonin was provided as a topical hair tonic. Non active ingredients included ethanol, benzyl alcohol, glycerin, and purified water.

The addition of 0.01% tretinoin and 0.1% latanoprost to a preparation containing 5% minoxidil, 0.25% finasteride and 0.1% melatonin was also made. However, despite being effective, the formulation was found to be more irritant to the scalp than the 10% minoxidil, 0.25% finasteride and 0.1% melatonin formulation. Therefore, the 10% minoxidil, 0.25% finasteride and 0.1% melatonin preparation was selected for use in the examples.

Six patients with androgenetic alopecia (five female patients (patients 1, 3, 4, 5 and 6 in Table 1) and one male patient (patient 2 in Table 1)) were recruited to the study. All had failed to respond to at least 6 months of continued standard topical 5% minoxidil treatment, twice daily. Three control patients (patients 7 to 9) received PRF treatment alone without formulation as shown in Table 1.

Baseline medical assessment, including a full history and examination were performed on all patients. Examination included trichoscopy and clinical photography. Scaling of the severity of androgenetic alopecia were also carried out in accordance with the Ludwig scale (for female patients) and the Norwood-Hamilton scale (for male patients). A dermatology quality of life index (DLQI) was also taken to assess the impact of androgenetic alopecia on the patient's quality of life. In order to be objective about the degree of hair loss, these clinical hair loss scales have been made and are used by clinicians. The scales can also be used at follow up to objectively document improvement in alopecia.

TABLE 1
Six patients with androgenetic alopecia recruited and started on topical hair tonic
(patients 1-6), following by up to 4 treatments of PRF (4 weeks apart). All patients
saw a 1 or 2 point improvement in their clinical hair scores at 3 month follow up.

| | | | | BASELINE | 3 MONTHS | 6 MONTHS |
|---|---|---|---|---|---|---|
| | | Tonic started | Number of iPRF | Ludwig | Ludwig | Ludwig |
| Patient | Sex, | 1 month prior | treatments | Score (F)/ | Score (F)/ | Score (F)/ |
| number | Age | to iPRF | (4 weeks apart) | Norwood (M) | Norwood (M) | Norwood (M) |
| 1 | F, 25 | 5% minoxidil, | 4 | I | 0 | n/a |
| | | 0.25% finasteride, | | | | |
| | | 0.1% melatonin | | | | |
| 2 | M, 21 | 10% minoxidil, | 3 | III | I | n/a |
| | | 0.25% finasteride, | | | | |
| | | 0.1% melatonin | | | | |
| 3 | F, 67 | 0.25% finasteride, | 4 | III | II | n/a |
| | | 0.1% melatonin | | | | |
| 4 | F, 50 | 0.25% finasteride, | 4 | I | 0 | n/a |
| | | 0.1% melatonin | | | | |
| 5 | F, 42 | 10% minoxidil, | 4 | II | 0 | 0 |
| | | 0.25% finasteride, | | | | |
| | | 0.1% melatonin | | | | |
| 6 | F, 61 | 5% minoxidil, | 2 | III | II | n/a |
| | | 0.25% finasteride, | | | | |
| | | 0.1% melatonin | | | | |
| 7 | M, 43 | none | 4 | II | II | II |
| 8 | F, 74 | none | 3 | II | II | n/a |
| 9 | F, 56 | none | 3 | II | II | n/a |

Three patients (patients 7-9) were controls where they received PRF treatment only, without the topical hair tonic.

Patients 3 and 4 of Table 1 used 1 ml of 0.25% finasteride and 0.1% melatonin daily at night time one month prior to PRF treatment.

Patients 2 and 5 of Table 1 used 1 ml of 10% minoxidil, 0.25% finasteride and 0.1% melatonin solution daily at night time one month prior to PRF treatment.

Patients 1 and 6 of Table 1 used 1 ml of 5% minoxidil, 0.25% finasteride and 0.1% melatonin solution daily at night time one month prior to PRF treatment.

Control patients 7 to 9 received only PRF treatment and did not receive any formulation treatment.

PRF is a second-generation platelet-rich plasma where autologous platelets are present in a complex fibrin matrix to accelerate healing of tissue. The iPRF Choukroun method is used.

A sample of blood is taken from the subject (in the examples relating to the present invention, 4 vials of blood are taken) and centrifuged (for example using a centrifuge from 5 minutes at 1200 revolutions per minute) to provide three layers consisting of a top layer of platelet poor plasma, middle layer of platelet rich fibrin clot and a bottom layer of red blood cells. The platelet rich fibrin layer may then be removed and the layer of red blood cells detached.

Once the PRF has been collected, 10 mls of PRF in 0.1 ml aliquots are then injected into the areas of hair thinning.

The hair tonic formulation is started 1 month prior to starting the first PRF, and 4 sessions of PRF are done 4 weeks apart.

Patients were then followed up after 3 months and then 6 months. All patients 1 to 6 reported hair growth and thickening of their hair. Objectively, clinical assessment revealed reduced hair follicle miniaturisation and at least 1 scale improvement in hair loss scale for all patients. Significant improvement in DLQI index was also measured for all patients. No adverse effects were reported from any of these patients.

Patients 3 and 4 of Table 1 used 1 ml of 0.25% finasteride and 0.1% melatonin daily at night time one month prior to PRF treatment (four PRF treatments, four weeks apart). Patients 3 and 4 experienced a one point improvement in their clinical hair scores at the three month follow up after treatment.

Patients 2 and 5 of Table 1 used 1 ml of 10% minoxidil, 0.25% finasteride and 0.1% melatonin solution daily at night time one month prior to PRF treatment. Patient 2 had three PRF treatments spaced four weeks apart, and experienced a two point improvement in their clinical hair score at the three month follow up after treatment. Patient 5 had four PRF treatments spaced four weeks apart, and experienced a two point improvements in their clinical hair score at the three month follow up after treatment.

Patients 1 and 6 of Table 1 used 1 ml of 5% minoxidil, 0.25% finasteride and 0.1% melatonin solution daily at night time one month prior to PRF treatment. Patient 1 had four PRF treatments spaced four weeks apart, and experienced a one point improvement in their clinical hair score at the three month follow up after treatment. Patient 6 had two PRF treatments spaced four weeks apart, and experienced a one point improvement in their clinical hair score at the three month follow up after treatment.

Control patients 7 to 9 received only PRF treatment and did not receive any formulation treatment. Control patient 7 received four treatments of PRF spaced four weeks apart, and experienced no improvement in their clinical hair score at the three month follow up after treatment. Control patients 8 and 9 received three treatments of PRF spaced four weeks apart, and experienced no improvement in their clinical hair score at the three month follow up after treatment.

No patients experienced paradoxical shedding.

There was found to be considerably fast visible hair growth after 6 to 8 weeks.

Hair growth results using (a) finasteride and melatonin formulation or (b) minoxidil, finasteride and melatonin formulation in the form of a tonic every night in conjunction with a course of platelet rich fibrin (PRF) are considerably quicker than when using the minoxidil, finasteride and melatonin formulation alone or PRF treatment alone.

### Example 2

Trials including five female patients using a treatment protocol of (a) finasteride and melatonin to eyebrows and (b) finasteride, melatonin and latanoprost to eyelashes, with the formulation in the form of a tonic every night have been conducted.

A formulation of 0.25% finasteride and 0.1% melatonin according to the present invention is a potent topical treatment to increase hair growth and/or reduce hair thinning in eyebrows.

A formulation of 0.25% finasteride, 0.1% melatonin and 0.005% latanoprost according to the present invention is a potent topical treatment to increase hair growth and/or reduce hair thinning in eyelashes.

A formulation of 0.25% finasteride, 0.1% melatonin and 0.001% latanoprost according to the present invention is a potent topical treatment to increase hair growth and/or reduce hair thinning in eyelashes.

The formulation of 0.25% finasteride and 0.1% melatonin was provided as a topical eyebrow tonic.

The formulation of 0.25% finasteride, 0.1% melatonin and 0.005% latanoprost was provided as a topical eyelash tonic.

The formulation of 0.25% finasteride, 0.1% melatonin and 0.001% latanoprost was provided as a topical eyelash tonic.

TABLE 2
Five patients were recruited and started on topical eyebrow/eyelash tonic (patients 1-5).

| | | | | | LASHES | LASHES |
|---|---|---|---|---|---|---|
| | | | BROWS | BROWS | 3 MONTH | 3 MONTHS |
| | | | BASELINE | 3 MONTHS | LENGTH | THICKNESS |
| | | | (% hair follicle | (% hair follicle | (shorter, no | (thinner, no |
| | | | minaturisation). | minaturisation). | difference, | difference, |
| Patient | Sex, | | Analysis of | Analysis of | longer). Patient | thicker). Patient |
| number | Age | Tonic | dermoscopy images. | dermoscopy images. | reported outcome. | reported outcome. |
| 1 | F, 54 | 0.25% finasteride, | 48 | 20 | Longer | Thicker |
| | | 0.1% melatonin | | | | |
| | | (to eyebrows) | | | | |
| | | 0.25% finasteride, | | | | |
| | | 0.1% melatonin, | | | | |
| | | 0.005% (to eyelashes) | | | | |
| 2 | F, 59 | 0.25% finasteride, | 30 | 17 | Longer | No difference |
| | | 0.1% melatonin | | | | |
| | | (to eyebrows) | | | | |
| | | 0.25% finasteride, | | | | |
| | | 0.1% melatonin, | | | | |
| | | 0.005% (to eyelashes) | | | | |
| 3 | F, 48 | 0.25% finasteride, | 78 | 60 | No difference | Thicker |
| | | 0.1% melatonin | | | | |
| | | (to eyebrows) | | | | |
| | | 0.25% finasteride, | | | | |
| | | 0.1% melatonin, | | | | |
| | | 0.001% (to eyelashes) | | | | |
| 4 | F, 64 | 0.25% finasteride, | 65 | 32 | Longer | Thicker |
| | | 0.1% melatonin | | | | |
| | | (to eyebrows) | | | | |
| | | 0.25% finasteride, | | | | |
| | | 0.1% melatonin, | | | | |
| | | 0.005% (to eyelashes) | | | | |
| 5 | F, 57 | 0.25% finasteride, | 57 | 41 | Longer | Thicker |
| | | 0.1% melatonin | | | | |
| | | (to eyebrows) | | | | |
| | | 0.25% finasteride, | | | | |
| | | 0.1% melatonin, | | | | |
| | | 0.005% (to eyelashes) | | | | |

Patients 1, 2, 4 and 5 of Table 2 used 0.25% finasteride and 0.1% melatonin on their eyebrows, and 0.25% finasteride, 0.1% melatonin and 0.005% latanoprost on their eyelashes. The tonic was applied daily at night time, with 2 drops of the tonic soaked onto cotton buds and applied to where the hair follicles emerge from the skin.

Patient 3 of Table 2 used 0.25% finasteride and 0.1% melatonin on their eyebrows, and 0.25% finasteride, 0.1% melatonin and 0.001% latanoprost on their eyelashes. The tonic was applied daily at night time, with 2 drops of the tonic soaked onto cotton buds and applied to where the hair follicles emerge from the skin.

Patients were then followed up after 3 months. Following treatment to eyebrows, all patients saw a reduction in % hair follicle minaturisation. Following treatment to eyelashes, four patients (patients 1, 2, 4 and 5) saw an increase in eyelash length and four patients (1, and 3 to 5) saw an increase in eyelash thickness.

1. A cosmetic method of increasing hair thickness, increasing hair growth, reducing hair thinning and/or reducing hair loss in a subject that has received or will subsequently or concurrently receive platelet rich fibrin (PRF) comprising administering a formulation comprising 0.01 wt % to 1 wt % finasteride and 0.01 wt % to 1 wt % melatonin to the scalp of the subject.

2. Use of a formulation comprising 0.01 wt % to 1 wt % finasteride and 0.01 wt % to 1 wt % melatonin to increase hair thickness, increase hair growth, reduce hair thinning and/or reduce hair loss in a subject that has received or will subsequently or concurrently receive platelet rich fibrin (PRF).

3. A formulation comprising, 0.01 wt % to 1 wt % finasteride and 0.01 wt % to 1 wt % melatonin for the treatment of hair loss in a subject that has received or will subsequently or concurrently receive platelet rich fibrin (PRF).

4. The cosmetic method, use and/or formulation for use according to any one of claims 1 to 3, wherein the hair loss is caused by androgenetic alopecia.

5. The cosmetic method, use and/or formulation for use according to any one of claims 1 to 4, wherein the formulation further comprises 1 wt % to 12.5 wt % minoxidil.

6. The cosmetic method, use and/or formulation for use according to claim 5, wherein the formulation further comprises 2 wt % to 11 wt % minoxidil.

7. The cosmetic method, use and/or formulation for use according to any one of claims 1 to 6, wherein the formulation comprises 0.1 wt % to 1 wt % finasteride.

8. The cosmetic method, use and/or formulation for use according to any one of claims 1 to 7, wherein the formulation comprises 0.05 wt % to 0.5 wt % melatonin.

9. The cosmetic method, use and/or formulation for use according to any one of claims 1 to 8, wherein the formulation further comprises a prostaglandin analogue at an amount between 0.0005 wt % to 5 wt %

10. The cosmetic method, use and/or formulation for use according to claim 9, wherein the prostaglandin analogue is selected from the group consisting of: travoprost, bimatoprost, tafluprost and unoprostone.

11. The cosmetic method, use and/or formulation for use according to claim 9, wherein the prostaglandin analogue is latanoprost.

12. The cosmetic method, use and/or formulation for use according to any one of claims 1 to 11, wherein the formulation further comprises a retinoic acid at an amount between 0.01 wt % to 2 wt %.

13. The cosmetic method, use and/or formulation for use according to claim 12, wherein the retinoic acid is tretinoin.

14. The cosmetic method, use and/or formulation for use according to any one of claims 1 to 13 wherein the formulation is in the form of an ointment, lotion and/or spray for topical administration.

15. The cosmetic method of any one of claims 1 to 14 wherein the method further comprises administering PRF to the scalp of the subject.

16. A cosmetic method of increasing hair thickness, increasing hair growth, reducing hair thinning and/or reducing hair loss of the eyebrows and/or eyelashes of a subject comprising administering a formulation comprising 0.01 wt % to 1 wt % finasteride and 0.01 wt % to 1 wt % melatonin to the eyebrows and/or eyelashes of the subject.

17. Use of a formulation comprising 0.01 wt % to 1 wt % finasteride and 0.01 wt % to 1 wt % melatonin to increase hair thickness, increase hair growth, reduce hair thinning and/or reduce hair loss of the eyebrows and/or eyelashes in a subject.

18. A formulation comprising, 0.01 wt % to 1 wt % finasteride and 0.01 wt % to 1 wt % melatonin for the treatment of hair loss of the eyebrows and/or eyelashes of a subject.
